(19) United States
(12) Patent Application Publication
(10) Pub. No.: US 2023/0217919 A1
(43) Pub. Date: Jul. 13, 2023

(54) COMPOSITION, AND PROCESS FOR COATING SEED MATERIAL WITH THE COMPOSITION

(71) Applicant: IPL BIOLOGICALS LIMITED, Gurugram (IN)

(72) Inventor: Vimala PRAKASH, Gurugram (IN)

(21) Appl. No.: 17/910,392

(22) PCT Filed: Mar. 11, 2021
(86) PCT No.: PCT/IN2021/050244

§ 371 Date: Sep. 9, 2022

(30) Foreign Application Priority Data
Mar. 11, 2020 (IN) 202011010469

(51) Int. Cl. A01N 25/24; A01N 37/22; A01N 43/56; A01N 43/88; A01N 25/10; A01N 63/22; A01N 63/20

(52) U.S. Cl. CPC A01N 25/24; A01N 37/22; A01N 43/56; A01N 43/88; A01N 25/10; A01N 63/22; A01N 63/20

(57) ABSTRACT

The present disclosure relates to a biosticker composition comprising: a) at least one polymer having a weight percentage in a range of 1-10% with respect to the composition; b) at least one starch having a weight percentage in a range of 2-15% with respect to the composition; c) at least one organic acid having a weight percentage in a range of 0.01-1% with respect to the composition; and d) at least one solvent having a weight percentage in a range of 1-10% with respect to the composition. The method of preparing the biosticker composition is also disclosed herein. The present disclosure also discloses a biocoat composition comprising the biosticker composition, at least one microbial culture, and at least one pesticide.

### FIELD OF THE INVENTION

The subject of the present disclosure broadly belongs to the field of agricultural science, and in particular, discloses a composition and a process for coating of seed material using the composition.

### BACKGROUND OF THE INVENTION

As per the data published in the Food and agriculture organization of the United Nations(http://www.fao.org/faostat/enNdata/EL/visualize,http://www.fao.org/faostat/en/#data/QV), agriculture is being practiced on more than one-third of the Earth's land. The total net production value is around 2.6×10⁹ USD. The agriculture sector is of paramount importance in providing jobs to millions of people around the globe. Apart from the sustainability provided by the agriculture sector, it also provides many methods to improve human well-being. Therefore, research is continuously required for improving agricultural methods and related technology with the help of increasing human knowledge. The integrated approach has been practiced by scientists, breeders, and policymakers around the globe to provide effective measures to sustain the agricultural domain. One of the objectives is to increase the agricultural yield that would ultimately feed millions of people around the globe.

In an effort to eradicate the ugly spot of hunger from the face of the humanity, there is an evident need to significantly increase the production and supply of food by integrating different elements and strengthening the plant breeding tools for crop improvements (Beddington et al. (2012). The role for scientists in tackling food insecurity and climate change. Agric. Food Sec. 1, 10). In pursuit of providing an increased yield, one of the areas which have shown potential success is the treatment of seed material to show increased resistance to pathogens and enhance survivability. Seed coating is becoming a popular method of improving the quality, plantability and ease of handling of seeds, ultimately leading to more efficient farming and improved yields. Further, it also has reduced major risks compared to other farming practices as it includes direct application of plant protection products on seeds per se, which eliminates the chances of direct human contact to harsh antimicrobials or insecticides. Such methods also dramatically reduce dusting-off of plant protection substances to the environmental surroundings, thereby leading to healthy and effective farming practices. However, the conventionally available methods for the coating of seeds are generally based on harsh chemical compounds which may either be toxic or non-biodegradable. The repeated use of synthetic chemical compounds like synthetic insecticides in the seed coating compositions also elevates the chances of resistance development in the insects or other target strains.

US20110105333A1 relates to seed treatment compositions comprising active ingredient and hyperbranched polymer, wherein the hyperbranched polymer is used as a binder to promote adhesion of the active ingredient(s) to the seeds. The application also relates to methods of treating seed with the said composition.

U.S. Pat. No. 4,251,952A relates to coated plant seed comprising a seed having a coating thereon comprising of polymeric material in direct contact with the normally exposed seed surface; the improvement comprising employing as such coating an intimate mixture of sugar and water insoluble polymeric material. The application further relates to the coating of plant seeds, including compositions and method therefor.

EP2308297A1 relates to an agrochemical composition suitable for seed treatment comprising herbicide and an adhesive polymer, and method of application thereof.

Despite of the ongoing extensive research in the present field of art to come up with an effective, harmless and affordable seed coating composition, there still lies a major gap between the conventionally available seed coat compositions and an ideal composition which may be help in advancing the aspects including germination, seedling vigour, nutrient uptake, and growth of the plant developing from the coated seed. Therefore, there exists a dire need in the present field of art demanding more focused research for the development of non-toxic, economical, and effective seed coat composition.

### SUMMARY OF THE INVENTION

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

In an aspect of the present disclosure, there is provided a composition comprising: (a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (b) at least one starch having a weight percentage in a range of 2-15% with respect to the composition; (c) organic acid having a weight percentage in a range of 0.01-1% with respect to the composition, wherein the organic acid is selected from the group consisting of citric acid, tartaric acid, ascorbic acid and combinations thereof; and (d) at least one solvent having a weight percentage in a range of 1-10% with respect to the composition, wherein the solvent is selected from the group consisting of glycerol, propylene glycol, and combinations thereof.

In another aspect of the present disclosure, there is provided a composition comprising: a) polyvinyl alcohol having a weight percentage in the range of 2-10% with respect to the composition; b) corn starch having a weight percentage in the range of 2-10% with respect to the composition; c) citric acid having a weight percentage in the range of 0.1-0.91% with respect to the composition; d) glycerol having a weight percentage in the range of 2-10% with respect to the composition; e) gum acacia having a weight percentage in the range of 2-10% with respect to the composition; f) xanthan gum having a weight percentage in the range of 2-10% with respect to the composition; and g) polyethylene glycol having a weight percentage in the range of 2-10% with respect to the composition.

In another aspect of the present disclosure, there is provided a process for preparing the composition as disclosed herein, said process comprising: a) contacting at least one polymer with water to obtain a first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) contacting the first mixture with at least one starch to obtain a second mixture, wherein the starch having a weight percentage in a range of 2-15% with respect to the composition is selected from the group consisting of corn starch, wheat starch, rice starch and combinations thereof; c) mixing the second mixture with at least one acid to obtain a third mixture, wherein the organic acid having a weight percentage in a range of 0.01-1% with respect to the composition is selected from the group consisting of citric acid, tartaric acid, ascorbic acid and combinations thereof; and d) contacting the third mixture with at least one solvent to obtain the composition, wherein the solvent having a weight percentage in a range of 1-10% with respect to the composition is selected from the group consisting of glycerol, propylene glycol, and combinations thereof, and wherein the composition has a pH in a range of 2-5.

In another aspect of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide.

In another aspect of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; and ii) at least one microbial culture.

In another aspect of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; and ii) adding the biocoat composition to a seed material followed by drying for coating the seed material, wherein adding the biocoat composition to the seed material in a volume in the range of 5 ml/1000 g-100 ml/1000 g seed.

In another aspect of the present disclosure, there is provided a process for coating a seed material, said process comprising: i) mixing the composition comprising: a) at least one polymer having a weight percentage in a range of 2-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-10% with respect to the composition; c) at least one organic acid having a weight percentage in a range of 0.1-0.91% with respect to the composition, wherein the organic acid is selected from the group consisting of citric acid, tartaric acid, ascorbic acid and combinations thereof; d) at least one solvent having a weight percentage in a range of 2-10% with respect to the composition, wherein the solvent is selected from the group consisting of glycerol, propylene glycol, and combinations thereof; e) gum acacia having a weight percentage in the range of 2-10% with respect to the composition; f) xanthan gum having a weight percentage in the range of 2-10% with respect to the composition; g) polyethylene glycol having a weight percentage in the range of 2-10% with respect to the composition; with the microbial culture and the pesticide to obtain the biocoat composition; and ii) adding the biocoat composition to a seed material followed by drying for coating the seed material, wherein adding the biocoat composition to the seed material in a volume in the range of 5 ml/1000 g-100 ml/1000 g seed.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “combinations thereof” relates to all sorts of combinations possible from the mentioned list of components, for example, “A” can be selected from the group of B, C, D, E, and combinations thereof, so, “A” here can be C or C and D or C and E, etc.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight percentage range of 1-5% should be interpreted to include not only the explicitly recited limits of about 1% to about 5%, but also to include sub-ranges, such as 2-4%, 2.5-3.5% and so forth, for example.

The term “at least one” is used to mean one or more and thus includes individual components as well as mixtures/combinations.

The term “MTCC” is the abbreviation of the Microbial Type Culture Collection, a national facility established in 1986 is funded jointly by the Department of Biotechnology (DBT) and the Council of Scientific and Industrial Research (CSIR), Government of India. MTCC has established itself as a distinguished culture collection centre for microbial resources in India. It is an affiliate member of the World Federation for Culture Collections (WFCC) and is registered with the World Data Centre for Microorganisms (WDCM). The main objectives of this national facility are to act as a depository, to supply authentic microbial cultures and to provide related services to the scientists working in research institutions, universities and industries.

The term “ITCC” is the abbreviation of the Indian Type Culture Collection, a national depositary authority to deposit strains of fungal and bacterial cultures with affiliation to World Federation for Culture Collections (WFCC). The main objectives of ITCC are to act as repositories for fungal and bacterial cultures, to provide services viz., supply of authentic fungal and bacterial cultures and identification of fungi and bacteria to technocrats and scientists working in research Institutions, Universities and Industries.

The term “at least one polymer” includes all the polymers which can be included for coating a seed material. One example is polyvinyl alcohol which has been exemplified, although other polymers can also be considered.

The term “at least one starch” is intended to include all different types of starch which are well-known to be used in the practices to which the present disclosure belongs. The starch may be selected from the group consisting of corn starch, wheat starch, rice starch, potato starch, cassava starch, food-derived starch, and combinations thereof, in accordance with the present disclosure.

The term “at least one solvent” is intended to include all different types of solvents for preparing a coating material as disclosed in the present disclosure. As per the present disclosure, the solvent may be selected from the group of glycerol, propylene glycol, and combinations thereof.

The term “at least one organic acid” is intended to include all types of organic acid which can be included for preparing the coating material as disclosed herein. The acid may be selected from the group consisting of citric acid, tartaric acid, ascorbic acid, and combinations thereof, in accordance with the present disclosure.

The term “microbial culture” is intended to cover all the microbial culture which can be exploited for coating the seed material. The present disclosure covers a few of them including Rhizobium spp, Azospirillum spp, Azotobacter spp, Bacillus subtilis, Phosphate solubilizing bacteria, Trichoderma viride, Paecilomyces spp and Pochonia spp, however, the list is not a limiting one and other microbial cultures are also to be considered within the scope of the present disclosure.

The term “seed material” or “seed” or “seedling” relates to seed from a single plant species, a mixture of seed from multiple plant species, or a seed blend from various strains within a plant species intended to be coated for their significance in terms of their seedling vigour, germination percentage, nutrient uptake, and agricultural produce. The seeds as mentioned herein intends to cover the seeds of plant varieties including, but not limited to cereals, millets, pulses, oil seeds, fibre cops, vegetable crops, sugar crops, and forage crops. The cereals are selected form the group consisting of rice (Oryza sativa), wheat, bread wheat (Triticum aestivum), corn (Zea mays), maize (Zea mays), barley (Hordeum vulgare); and the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis); and the fibre crops are selected form the group consisting of cotton (Gossypium spp.); and jute (Corchorus olitorius); and the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa); and the sugar crops are selected from the group consisting of sweet sorghum (Sorghum spp.), and sugar beet (Beta vulgaris); and the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala).

The term “seed coating” denotes any process that endows the outer surfaces of the seeds partially or completely with a layer or layers of non-plant material. In context with the present disclosure, the said non-plant material relates to the biocoat composition for the use in coating of seed material.

The term “biosticker” refers to the composition as disclosed in the present disclosure, wherein the said composition comprises of: (a) at least one polymer having a weight percentage in a range of 1-10% with respect to the composition; (b) at least one starch having a weight percentage in a range of 2-15% with respect to the composition; (c) at least one organic acid having a weight percentage in a range of 0.01-1% with respect to the composition; and (d) at least one solvent having a weight percentage in a range of 1-10% with respect to the composition. The biosticker composition may further comprise of the excipients including, gum acacia, xanthum gum, polyethylene glycol, natural or synthetic colour, and combinations thereof, wherein gum acacia has weight percentage in the range of 0.5-10% with respect to the composition, xanthan gum has a weight percentage in the range of 0.5-10% with respect to the composition, polyethylene glycol has a weight percentage in the range of 0.5-10% with respect to the composition, and natural or synthetic colour has a weight percentage in the range of 0.05-0.1% with respect to the composition.

The term “biocoat” or “seed coating composition” refers to the composition as disclosed in the present disclosure for the coating of seed material, wherein the said composition comprises of: a) the biosticker composition as obtained in Example 2 of the present disclosure; b) at least one microbial culture; and c) at least one pesticide, wherein the microbial culture has microbial load in the range of 10⁷-10¹⁰ in 100 ml of the biocoat composition. The biocoat composition may also relate to a composition comprising, a) the biosticker composition as obtained in Example 2 of the present disclosure; b) at least one microbial culture, wherein the microbial culture has microbial load in the range of 10⁷-10¹⁰ in 100 ml of the biocoat composition.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background of the present disclosure, seed treatment or seed coating is becoming one of the most viable alternatives to the traditional broadcast spraying of insecticides or similar processes to support the germination and growth of large variety of crops. However, the use of chemicals and synthetic compounds exhibiting toxic and non-biodegradable properties in the conventionally available seed coating compositions poses higher risk of development of side effects to both humans and the environmental conditions. Many of the conventional seed coating methods include the use of non-biodegradable synthetic compounds which directly or indirectly hampers the idea of sustainable agriculture practices. Resultingly, to fulfill the dire need for an effective, safe and biodegradable seed coating composition, the present disclosure discloses a biosticker composition comprising: (a) at least one polymer having a weight percentage in a range of 1-10% with respect to the composition; (b) at least one starch having a weight percentage in a range of 2-15% with respect to the composition; (c) at least one organic acid having a weight percentage in a range of 0.01-1% with respect to the composition; and (d) at least one solvent having a weight percentage in a range of 1-10% with respect to the composition. Further, the present disclosure relates to a biocoat composition comprising: (a) biosticker composition; (b) at least one microbial culture; and c) at least one pesticide, wherein the microbial culture has microbial load is in the range of 10⁷-10¹⁰ in 100 ml of the biocoat composition, for the use as seed coating composition. The chemical pesticides used in the biocoat composition as disclosed herein imparts disease protection to the coated seed material, and alongside, the microbial culture provides necessary microbial presence around the coated seed for the nutritional benefits and optimal growth. Unlike the conventional methods which may only provide protection from disease causing organisms to the seeds, the biaocoat composition as disclosed herein additionally provides enhanced germination percentage, nutrient uptake, seedling vigour, growth and lowered damping off in coated seeds on comparison to non-coated seeds, thereby exhibiting synergistic effect as compared to the non-coated seeds.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof.

In an embodiment of the present disclosure, there is provided a composition comprising: a) at least one polymer having a weight percentage in a range of 1-9% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the at least polymer has a weight percentage in the range of 1-8% with respect to the composition. In yet another embodiment of the present disclosure, the at least polymer has a weight percentage in the range of 1-7% with respect to the composition. In one another embodiment of the present disclosure, the at least polymer has a weight percentage in the range of 1-6% with respect to the composition. In an alternate embodiment of the present disclosure, the at least polymer has a weight percentage in the range of 1-5% with respect to the composition. In certain non-limiting examples, the weight percentage of the polymer with respect to the composition can be 1%, 2%, 3%, 4%, 5%, 6%, 7%, 8%, 9%, and 9.5%.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the polymer is selected from the group consisting of polyvinyl alcohol, polyvinyl acetate, and polyvinyl pyrrolidones. In yet another embodiment of the present disclosure, the polymer is polyvinyl alcohol.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-14% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the at least one starch has a weight percentage in a range of 2-12% with respect to the composition. In yet another embodiment of the present disclosure, the at least one starch has a weight percentage in a range of 4-10% with respect to the composition. In one embodiment of the present disclosure, the at least one starch has a weight percentage in a range of 4-8% with respect to the composition. In an alternate embodiment of the present disclosure, the at least one starch has a weight percentage in a range of 5-7% with respect to the composition. In certain non-limiting examples, the weight percentage of the starch with respect to the composition can be 2%, 3%, 4%, 5%, 6%, 7%, 8%, 9%, 10%, 12%, 14%, and 15%.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition, wherein the starch is selected from the group consisting of corn starch, wheat starch, and rice starch; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the starch is corn starch.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition, wherein the starch is selected from the group consisting of corn starch, wheat starch, and rice starch; c) organic acid having a weight percentage in a range of 0.01-0.9% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the organic acid has a weight percentage in a range of 0.014-0.8% with respect to the composition. In yet another embodiment of the present disclosure, the organic acid has a weight percentage in a range of 0.015-0.7% with respect to the composition. In one another embodiment of the present disclosure, the organic acid has a weight percentage in a range of 0.015-0.6% with respect to the composition. In certain non-limiting examples, the weight percentage of the organic acid with respect to the composition can be 0.01%, 0.015%, 0.02%, 0.05%, 0.1%, 0.15%, 0.25%, 0.5%, 0.7%, 0.9%, and 1%.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition, wherein the starch is selected from the group consisting of corn starch, wheat starch, and rice starch; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the starch is corn starch. In another embodiment of the present disclosure, the organic acid is citric acid.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-9% with respect to the composition, wherein the solvent is selected from the group consisting of glycerol, propylene glycol, and combinations thereof. In another embodiment of the present disclosure, the at least one solvent has a weight percentage in a range of 2-8% with respect to the composition. In yet another embodiment of the present disclosure, the at least one solvent has a weight percentage in the range of 3-7% with respect to the composition. In certain non-limiting examples, the weight percentage of the solvent with respect to the composition can be 1%, 2%, 4%, 6%, 8%, and 10%.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof. In another embodiment of the present disclosure, the solvent is glycerol.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) at least one excipient.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) at least one excipient selected from the group consisting of gum acacia, xanthum gum, polyethylene glycol, natural or synthetic colour, and combinations thereof.

In an embodiment of the present disclosure, there is provided a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) at least one excipient selected from the group consisting of gum acacia has a weight percentage in the range of 0.5-10% with respect to the composition, xanthan gum has a weight percentage in the range of 0.5-10% with respect to the composition, polyethylene glycol has a weight percentage in the range of 0.5-10% with respect to the composition, and natural or synthetic colour has a weight percentage in the range of 0.05-0.1% with respect to the composition. In another embodiment of the present disclosure, the gum acacia has a weight percentage in the range of 1-10%, or 1-9%, or 2-8%, or 3-8%, or 4-7%, or 4.5-6.5% with respect to the composition, xanthan gum has a weight percentage in the range of 0.5-9%, or 1-9%, or 2-9%, or 3-8%, or 4-7% with respect to the composition, polyethylene glycol has a weight percentage in the range of 0.5-9%, or 1-8%, or 2-9%, or 3-8%, or 4-7%, or 4-6% with respect to the composition, and natural or synthetic colour has a weight percentage in the range of 0.05-0.09% with respect to the composition.

In an embodiment of the present disclosure, there is provided a composition comprising: a) polyvinyl alcohol having a weight percentage in the range of 2-10% with respect to the composition; b) corn starch having a weight percentage in the range of 2-10% with respect to the composition; c) citric acid having a weight percentage in the range of 0.1-0.91% with respect to the composition; d) glycerol having a weight percentage in the range of 2-10% with respect to the composition; e) gum acacia having a weight percentage in the range of 2-10% with respect to the composition; f) xanthan gum having a weight percentage in the range of 2-10% with respect to the composition; and g) polyethylene glycol having a weight percentage in the range of 2-10% with respect to the composition. In another embodiment of the present disclosure, polyvinyl alcohol has a weight percentage in the range of 2-9%, or 2-8%, or 3-7%, or 4-6% with respect to the composition; corn starch has a weight percentage in the range of 2-9%, or 3-8%, or 4-7%, with respect to the composition; c) citric acid having a weight percentage in the range of 0.1-0.9%, 0.2-0.8%, or 0.3-0.7%, or 0.4-0.6%, with respect to the composition; d) glycerol having a weight percentage in the range of 2-9%, or 3-8%, or 3-7%, or 3-6%, or with respect to the composition; e) gum acacia having a weight percentage in the range of 2-9%, or 2-8%, or 3-7%, with respect to the composition; f) xanthan gum having a weight percentage in the range of 2-9%, or 2-8%, 3-7%, or 3-6%, with respect to the composition; and g) polyethylene glycol having a weight percentage in the range of 2-9%, or 2-8%, or 3-7%, or 3-6%, with respect to the composition.

In an embodiment of the present disclosure, there is provided a process for preparing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof, said process comprising: (i) contacting at least one polymer with water to obtain a first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (ii) contacting the first mixture with at least one starch to obtain a second mixture, wherein the starch having a weight percentage in a range of 2-15% with respect to the composition is selected from the group consisting of corn starch, wheat starch, rice starch and combinations thereof; (iii) mixing the second mixture with at least one acid to obtain a third mixture, wherein the organic acid having a weight percentage in a range of 0.01-1% with respect to the composition is selected from the group consisting of citric acid, tartaric acid, ascorbic acid and combinations thereof; and (iv) contacting the third mixture with at least one solvent to obtain the composition, wherein the solvent having a weight percentage in a range of 1-10% with respect to the composition is selected from the group consisting of glycerol, propylene glycol, and combinations thereof, and wherein the composition has a pH in a range of 2-5. In another embodiment of the present disclosure, the composition has a pH in a range of 2-4.

In an embodiment of the present disclosure, there is provided a process for preparing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof, said process comprising: (i) contacting the at least one polymer with water is carried out at a temperature in the range of 80-95° C. to obtain the first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (ii) contacting the first mixture with the at least one starch is carried out at a temperature in the range of 80-85° C. to obtain the second mixture, wherein the starch having a weight percentage in a range of 2-15% with respect to the composition is selected from the group consisting of corn starch, wheat starch, rice starch and combinations thereof; (iii) mixing the second mixture with the at least one citric acid is carried out at a temperature in the range of 60-70° C. to obtain the third mixture, wherein the organic acid having a weight percentage in a range of 0.01-1% with respect to the composition is selected from the group consisting of citric acid, tartaric acid, ascorbic acid and combinations thereof; and (iv) contacting the third mixture with the at least one solvent is carried out at a temperature in the range of 50-60° C. to obtain the composition, wherein the solvent having a weight percentage in a range of 1-10% with respect to the composition is selected from the group consisting of glycerol, propylene glycol, and combinations thereof, and wherein the composition has a pH in a range of 2-5.

In an embodiment of the present disclosure, there is provided a process for preparing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; and d) at least one solvent having a weight percentage in a range of 1-10% with respect to the composition, wherein the solvent is selected from the group consisting of glycerol, propylene glycol, and combinations thereof, said process comprising: (i) contacting the at least one polymer with water is carried out at a temperature in the range of 82-90° C. to obtain the first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (ii) contacting the first mixture with the at least one starch is carried out at a temperature in the range of 82-84° C. to obtain the second mixture, wherein the starch having a weight percentage in a range of 2-15% with respect to the composition is selected from the group consisting of corn starch, wheat starch, rice starch and combinations thereof; (iii) mixing the second mixture with the at least one citric acid is carried out at a temperature in the range of 62-68° C. to obtain the third mixture, wherein the organic acid having a weight percentage in a range of 0.01-1% with respect to the composition is selected from the group consisting of citric acid, tartaric acid, ascorbic acid and combinations thereof; and (iv) contacting the third mixture with the at least one solvent is carried out at a temperature in the range of 52-58° C. to obtain the composition, wherein the solvent having a weight percentage in a range of 1-10% with respect to the composition is selected from the group consisting of glycerol, propylene glycol, and combinations thereof, and wherein the composition has a pH in a range of 2-5.

In an embodiment of the present disclosure, there is provided a process for preparing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof, and e) the excipient is selected from the group consisting of gum acacia, xanthum gum, polyethylene glycol, natural or synthetic colour, and combinations thereof, said process comprising: (i) contacting the at least one polymer with water is carried out at a temperature in the range of 80-95° C. to obtain the first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (ii) contacting the first mixture with the at least one starch is carried out at a temperature in the range of 80-85° C. to obtain the second mixture, wherein the starch having a weight percentage in a range of 2-15% with respect to the composition is selected from the group consisting of corn starch, wheat starch, rice starch and combinations thereof; (iii) mixing the second mixture with the at least one citric acid is carried out at a temperature in the range of 60-70° C. to obtain the third mixture, wherein the organic acid having a weight percentage in a range of 0.01-1% with respect to the composition is selected from the group consisting of citric acid, tartaric acid, ascorbic acid and combinations thereof; and (iv) contacting the third mixture with the at least one solvent is carried out at a temperature in the range of 50-60° C. to obtain the composition, followed by contacting the third mixture with the excipient selected from the group consisting of gum acacia, xanthum gum, polyethylene glycol, natural or synthetic colour, and combinations thereof, to obtain the composition, wherein the solvent having a weight percentage in a range of 1-10% with respect to the composition is selected from the group consisting of glycerol, propylene glycol, and combinations thereof, and wherein the composition has a pH in a range of 2-5.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide

In an embodiment of the present disclosure, there is provided a biocoat composition comprising: (i) a composition comprising: a) at least one polymer having a weight percentage in a range of 1-9% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide. In another embodiment of the present disclosure, the at least polymer has a weight percentage in the range of 1-8% with respect to the composition. In yet another embodiment of the present disclosure, the at least polymer has a weight percentage in the range of 1-7% with respect to the composition. In one another embodiment of the present disclosure, the at least polymer has a weight percentage in the range of 1-6% with respect to the composition. In an alternate embodiment of the present disclosure, the at least polymer has a weight percentage in the range of 1-5% with respect to the composition. In certain non-limiting examples, the weight percentage of the polymer with respect to the composition can be 1%, 2%, 3%, 4%, 5%, 6%, 7%, 8%, 9%, and 9.5%.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-14% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide. In another embodiment of the present disclosure, the at least one starch has a weight percentage in a range of 2-12% with respect to the composition. In yet another embodiment of the present disclosure, the at least one starch has a weight percentage in a range of 4-10% with respect to the composition. In one embodiment of the present disclosure, the at least one starch has a weight percentage in a range of 4-8% with respect to the composition. In an alternate embodiment of the present disclosure, the at least one starch has a weight percentage in a range of 5-7% with respect to the composition. In certain non-limiting examples, the weight percentage of the starch with respect to the composition can be 2%, 3%, 4%, 5%, 6%, 7%, 8%, 9%, 10%, 12%, 14%, and 15%.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-0.9% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide. In another embodiment of the present disclosure, the organic acid has a weight percentage in a range of 0.014-0.8% with respect to the composition. In yet another embodiment of the present disclosure, the organic acid has a weight percentage in a range of 0.015-0.7% with respect to the composition. In one another embodiment of the present disclosure, the organic acid has a weight percentage in a range of 0.015-0.6% with respect to the composition. In certain non-limiting examples, the weight percentage of the organic acid with respect to the composition can be 0.01%, 0.015%, 0.02%, 0.05%, 0.1%, 0.15%, 0.25%, 0.5%, 0.7%, 0.9%, and 1%.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-9% with respect to the composition, wherein the solvent is selected from the group consisting of glycerol, propylene glycol, and combinations thereof; ii) at least one microbial culture; and iii) at least one pesticide. In another embodiment of the present disclosure, the at least one solvent has a weight percentage in a range of 2-8% with respect to the composition. In yet another embodiment of the present disclosure, the at least one solvent has a weight percentage in the range of 3-7% with respect to the composition. In certain non-limiting examples, the weight percentage of the solvent with respect to the composition can be 1%, 2%, 4%, 6%, 8%, and 10%.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof; and iii) at least one pesticide.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof; and iii) at least one pesticide, wherein the microbial culture has microbial load in the range of 10⁷-10¹⁰ in 100 ml of the biocoat composition. In another embodiment of the present disclosure, the microbial culture has microbial load in the range of 10⁷-10⁸ in 100 ml of the biocoat composition.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide is selected from the group consisting of insecticides, fungicides, nematicide, herbicides, and combinations thereof.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide is selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide has a weight percentage in the range of 0.5-15% with respect to the composition. In another embodiment of the present disclosure, the at least one pesticide has a weight percentage in the range of 0.9-14% with respect to the composition. In yet another embodiment of the present disclosure, the at least one pesticide has a weight percentage in the range of 1-13% with respect to the composition. In one another embodiment of the present disclosure, the at least one pesticide has a weight percentage in the range of 1-10% with respect to the composition. In an alternate embodiment of the present disclosure, the at least one pesticide has a weight percentage in the range of 2-8% with respect to the composition.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture; and iii) at least one pesticide is selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin, and wherein the at least one pesticide has a weight percentage in the range of 1-10% with respect to the composition.

In an embodiment of the present disclosure, there is provided a biocoat composition comprising: i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; ii) at least one microbial culture selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof; and wherein the microbial culture has microbial load in the range of 10⁷-10¹⁰ in 100 ml of the biocoat composition; and iii) at least one pesticide is selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin, and wherein the at least one pesticide has a weight percentage in the range of 1-10% with respect to the composition.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture; and iii) at least one pesticide.

In an embodiment of the present disclosure, there is provided a biocoat composition comprising: (i) a composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture; and iii) at least one pesticide.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture wherein the microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof; and iii) at least one pesticide.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture wherein the microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof; and iii) at least one pesticide, wherein the microbial culture has microbial load in the range of 10⁷-10¹⁰ in 100 ml of the biocoat composition.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture; and iii) at least one pesticide selected from the group consisting of insecticides, fungicides, nematicide, herbicides, and combinations thereof.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture; and iii) at least one pesticide selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS, 2RS; 1RS, 2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin.

In an embodiment of the present disclosure, there is provided a biocoat composition comprising: (i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, wherein the microbial culture has microbial load in the range of 10⁷-10¹⁰ in 100 ml of the biocoat composition; and iii) at least one pesticide selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin, and wherein the pesticide has a weight percentage in the range of 0.5-15% with respect to the composition.

In an embodiment of the present disclosure, there is provided a biocoat composition comprising: (i) a composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, and wherein the microbial culture has microbial load in the range of 10⁷-10¹⁰ in 100 ml of the biocoat composition; and iii) at least one pesticide selected from the group consisting of insecticides, fungicides, nematicide, herbicides, and combinations thereof, wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin, and wherein the pesticide has a weight percentage in the range of 0.5-15% with respect to the composition.

In an embodiment of the present disclosure, there is provided a biocoat composition comprising: i) the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; b) at least one microbial culture.

In an embodiment of the present disclosure, there is provided a biocoat composition comprising: i) the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; b) at least one microbial culture selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof.

In an embodiment of the present disclosure, there is provided a biocoat composition comprising: i) the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; b) at least one microbial culture selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, wherein the microbial culture has microbial load in the range of 10⁷-10¹⁰ in 100 ml of the biocoat composition.

In an embodiment of the present disclosure, there is provided a biocoat composition comprising: (i) a composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture.

In an embodiment of the present disclosure, there is provided a biocoat composition comprising: (i) a composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof.

In an embodiment of the present disclosure, there is provided a biocoat composition comprising: (i) a composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition; ii) at least one microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, and wherein the microbial culture has microbial load in the range of 10⁷-10¹⁰ in 100 ml of the biocoat composition.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material, wherein adding the biocoat composition to the seed material in a volume in the range of 5 ml/1000 g-100 ml/1000 g seed. In another embodiment of the present disclosure, adding the biocoat composition to the seed material in a volume in the range of 10 ml/1000 g-90 ml/1000 g seed. In yet another embodiment of the present disclosure, adding the biocoat composition to the seed material in a volume in the range of 20 ml/1000 g-60 ml/1000 g seed.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material is done under sterile condition, wherein adding the biocoat composition to the seed material in a volume in the range of 5 ml/1000 g-100 ml/1000 g seed.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, and wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala).

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material, wherein adding the biocoat composition to the seed material in a volume in the range of 5 ml/1000 g-100 ml/1000 g seed, wherein the microbial culture has microbial load in the range of 10⁷-10¹⁰ in 100 ml of the biocoat composition.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material is done under sterile condition, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, and wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala), and wherein the microbial culture has microbial load in the range of 10⁷-10¹⁰ in 100 ml of the biocoat composition.

In an embodiment of the present disclosure, there is provided a process for coating a seed material, said process comprising: i) mixing the composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; with the microbial culture and the pesticide to obtain the biocoat composition; ii) adding the biocoat composition to a seed material followed by drying for coating the seed material is done under sterile condition, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, and wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala), and wherein the microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, and wherein the microbial culture has microbial load in the range of 10⁷-10⁸ in 100 ml of the biocoat composition, and wherein the pesticide is selected from the group consisting of insecticides, fungicides, nematicide, herbicides, and combinations thereof, and wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material material is done under sterile condition, wherein adding the biocoat composition to the seed material in a volume in the range of 5 ml/1000 g-100 ml/1000 g seed.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala).

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala), and wherein the microbial culture has microbial load in the range of 10⁷-10⁸ in 100 ml of the biocoat composition.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala), and wherein the microbial culture has microbial load in the range of 10⁷-10⁸ in 100 ml of the biocoat composition.

In an embodiment of the present disclosure, there is provided a process for coating a seed material, said process comprising: i) mixing the composition comprising: a) polyvinyl alcohol having a weight percentage in a range of 1-10% with respect to the composition; b) corn starch having a weight percentage in a range of 2-15% with respect to the composition; c) citric acid having a weight percentage in a range of 0.01-1% with respect to the composition; d) glycerol having a weight percentage in a range of 1-10% with respect to the composition; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition with the microbial culture and the pesticide to obtain the biocoat composition; and b) adding the biocoat composition to a seed material followed by drying for coating the seed material, wherein adding the biocoat composition to the seed material in a volume in the range of 5 ml/1000 g-100 ml/1000 g seed, wherein the seed material is selected from the group consisting of cereals, millets, pulses, oil seeds, fibre crops, vegetable crops, sugar crops, and forage crops, wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala), and and wherein the microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof, and wherein the microbial culture has microbial load in the range of 10⁷-10¹⁰ in 100 ml of the biocoat composition, and wherein the pesticide is selected from the group consisting of insecticides, fungicides, nematicide, herbicides, and combinations thereof, and wherein the insecticides are selected from the group consisting of Chlorpyriphos, Diazinon, Imidacloprid, Lindane, Permethrin, Tefluthrin, Thiamethoxam, and Cyantranilliprole, and wherein the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl, and wherein the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp., and wherein the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

The forthcoming section describes the biosticker composition and its further use in preparing the biocoat composition. The said biocoat composition as disclosed herein can be used for the coating of seeds before sowing. The biocoating composition as disclosed herein helps the seeds to elevate the germination percentage, seedling vigour of the coated seeds. It also helps in enhancing the mean root and shoot length of the coated seeds post germination. Further, the use of present biocoat composition also helps in minimizing the events of damping off under sick soil conditions. The biocoating of seeds also reduces the human and environmental contact with harmful agrochemicals. Therefore, the biosticker and biocoat compositions as disclosed herein provides qualitatively enhanced characteristics to the coated seeds in comparison to uncoated seeds.

### Example 1

### Biosticker Composition

In the present example, different components such as polyvinyl alcohol (polymer), corn starch (starch), citric acid (organic acid), glycerol (solvent), xanthan gum, gum acacia and polyethylene glycol were combined at different weight percentages to arrive at different compositions (biosticker compositions).

Table 1 shows seven biosticker compositions (C1-C7), wherein the weight percentages of the constituents of all said biosticker compositions are provided below. In each composition, the total volume was made up by adding water.

TABLE 1

| Biosticker | Polyvinyl | Corn | | | | | Polyethylene | |
|---|---|---|---|---|---|---|---|---|
| composition | alcohol | starch | Citric acid | Glycerol | Xanthan Gum | Gum Acacia | Glycol | |
| (Composition) | (Polymer) | (Starch) | (Organic acid) | (Solvent) | (Excipient) | (Excipient) | (Excipient) | Water |
| C 1 | 1% | 15% | 1% | 6% | 8% | 1% | 6% | 62% |
| C 2 | 2% | 10% | 0.5% | 10% | 6% | 3% | 10% | 58.5% |
| C 3 | 3% | 5% | 0.25% | 2% | 4% | 4% | 2% | 79.75% |
| C 4 | 5% | 7% | 0.10% | 1% | 3% | 6% | 1% | 76.9% |
| C 5 | 7% | 4% | 0.015% | 4% | 2% | 7% | 4% | 71.99% |
| C 6 | 10% | 2% | 0.01% | 8% | 1% | 8% | 8% | 62.99% |
| C7 | 10% | 15% | 0.1% | 5% | 8% | 8% | 10% | 43.1% |

In the biosticker compositions as shown in Table 1 above, the weight percentages of the constituents in C1 were, 1% polyvinyl alcohol, 15% corn starch, 1% citric acid, 6% glycerol, 8% xanthan gum, 1% gum acacia, 6% polyethylene glycol, and 62% water. Similarly, C 2, C 3, C 4, C 5, C 6 and C 7 has: a) polyvinyl alcohol in the weight percentage of 2%, 3%, 5%, 7%, 10% and 10% respectively; b) corn starch in the weight percentage of 10%, 5%, 7%, 4%, 2% and 15% respectively; c) citric acid in the weight percentage of 0.5%, 0.25%, 0.10%, 0.015%, 0.01%, 0.1% respectively; d) glycerol in the weight percentage of 10%, 2%, 1%, 4%, 8% and 5% respectively; e) xanthan gum in the weight percentage of 6%, 4%, 3%, 2%, 1% and 8% respectively; f) gum acacia in the weight percentage of 3%, 4%, 6%, 7%, 8% and 8% respectively; g) polyethylene glycol in the weight percentage of 10%, 2%, 1%, 4%, 8% and 10% respectively; and h) water in the weight percentage of 58.5%, 79.75%, 76.9%, 71.99%, 62.99% and 43.1% respectively.

The biotsicker composition further comprised of natural colors like orange red color (Procured from August Food Ingredients, Kerela), Marigold yellow (Procured from Symega Foods ingredients Limited, Kerela), and synthetic colour like Bright green (Procured from Ajanta Food Product Company, Solan, Himachal Pradesh) at a weight percentage in the range of 0.05-0.1% with respect to the composition.

The properties of the corn starch as used in the biosticker compositions (Table 1) in the present example are depicted in Table 2 of the present disclosure.

TABLE 2

| S. No. | Parameters | Specification | |
|---|---|---|---|
| 1. | Molecular weight | 692.7 | g/mol |
| 2. | Molecular formula | C27H48O20 | |
| 3. | Starch (on dry basis) Percent by | 98-99 | |
| | mass, Min | | |
| 4. | Moisture (% w/w) | 10-13 | |
| 5. | Cold Water Soluble (% on DB) | 0.50 | Max |
| 6. | Viscosity of 2% paste at 75' C. on | 37.0 | Min. |
| | Redwood viscometer No. 1 (Secs) | | |
| 7. | pH | 5.0-7.0 (25° C., 2% | |
| | | in solution) | |
| 8. | Bulk density (gm/ml) | 0.50 | in. |

The criticality of the present disclosure lies in the presence of the combination of the components such as polyvinyl alcohol (polymer), corn starch (starch), citric acid (organic acid), glycerol (solvent), xanthan gum, gum acacia and polyethylene glycol at the disclosed weight percentage range of 1-10%, 2-15%, 0.01-1%, 1-10%, respectively to arrive at the biosticker compositions. The biotsicker compositions was further used in the biocoating compositions.

It can also be contemplated that a person skilled in the art can arrive at the different biotsicker compositions having polymers like polyvinyl alcohol, polyvinyl alcohol copolymers, polyvinyl acetate, polyvinyl acetate copolymers, polyvinyl pyrrolidones, vinylidene chloride, vinylidene chloride copolymers, starch like corn starch, wheat starch, and rice starch, organic acid like citric acid, tartaric acid, and ascorbic acid, and solvent like glycerol, propylene glycol, when present within the disclosed weight percentage ranges.

### Example 2

### Process for Preparing the Biosticker Composition

The present example describes the process for preparing the biosticker composition of the present disclosure. The process for preparing the biosticker composition involves the step-wise addition of individual constituents at the disclosed weight percentages. The process of the present disclosure comprised the following steps: (i) the polymer was contacted with water at a temperature in the range of 80-95° C. to obtain a first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; (ii) the first mixture was contacted with at least one starch at a temperature in the range of 80-85° C. to obtain a second mixture, wherein the starch having a weight percentage in a range of 2-15% with respect to the composition is selected from the group consisting of corn starch, wheat starch, rice starch and combinations thereof; (iii) the second mixture was mixed with at least one acid at a temperature in the range of 60-70° C. to obtain a third mixture, wherein the organic acid having a weight percentage in a range of 0.01-1% with respect to the composition is selected from the group consisting of citric acid, tartaric acid, ascorbic acid and combinations thereof; and (iv) the third mixture was contacted with at least one solvent at a temperature in the range of 50-60° C. to obtain the biosticker composition, wherein the solvent having a weight percentage in a range of 1-10% with respect to the composition is selected from the group consisting of glycerol, propylene glycol, and combinations thereof, and wherein the composition has a pH in a range of 2-5. The process further comprised contacting the third mixture with the excipient selected from the group consisting of gum acacia, xanthan gum, polyethylene glycol, natural or synthetic colour, and combinations thereof, to obtain the biostciker composition having excipients.

Process for preparing Biosticker Composition C7: The present example provides the process for preparing the biosticker composition C7 (as shown in Table 1), For the purpose of preparing the composition C7, quantity sufficient distilled water was heated to a temperature range of 80-90° C. to which 10% of polyvinyl alcohol (polymer) was added at a rate of 10 g/minute with continuous stiffing to obtain a first mixture. The first mixture so obtained was milky white in color. While mixing the polymer with water, the clump formation can be avoided by adjusting the rate of addition of polyvinyl alcohol, which could be in the range of 6g/minute. However, the rate of addition can ranged between 6 to 20 g/minute. The temperature of the first mixture was then adjusted in the range of 80-85° C., followed by the addition of 15% of corn starch (starch) with respect to the composition to the first mixture at a rate of 20 g/minute to obtain a second mixture. The second mixture so obtained was milky white in color. In case of clump formation while mixing, the rate of addition of starch should be adjusted in a range of 15 to 30 g/minute to avoid clumping. The temperature of the second mixture was adjusted to a range of 50-60° C., followed by the addition of 0.1% citric acid (organic acid) to the second mixture at a rate of 12g/minute to obtain a third mixture. The rate of addition of citric acid to the second mixture could be in a range of to 20 g/minute. The temperature of the third mixture was then adjusted in the range of 56-60° C., followed by the addition of 5% glycerol (solvent) with respect to the composition to the third mixture at a rate of 10 ml/minute to obtain the biosticker composition of the present disclosure. The rate of addition of glycerol to the third mixture could be in a range of 10 to 50 ml/minute to obtain the biosticker composition. To the third mixture, the components such as xanthan gum, gum acacia and polyethylene glycol (excipients) were further added in the weight percentages of 8%, 8%, and 10%, respectively to obtain the final biosticker composition (C7). The biosticker composition obtained via aforementioned process was transparent to milky-white in color, and had a pH value in a range of 2-5. However, it can be contemplated that the pH value of the biotsicker composition can be 2, 3, 4, or 5.

Similarly, all the biosticker composition C1-C6 as per the Table 1 of Example 1 can be prepared by following the afore-mentioned process as mentioned above. However, it can be contemplated that the person skilled in the art can arrive at different biosticker compositions with the constituents in the disclosed weight percentages by performing the process as disclosed herein.

### Example 3

### Process for Preparing Biocoat Composition

The biosticker composition as mentioned in Example 1 was further used for preparing the biocoat composition. The present disclosure provides two types of biocoat compositions: (a) Biocoat composition having a combination of biosticker compositions and microbial culture; and (b) Biocoat composition having a combination of biosticker composition, microbial culture, and chemical pesticides. The biocoat composition was further used for coating the seeds.

Process for preparing the biocoat composition comprising the biosticker composition and microbial culture: The present example pertains to the process of preparing biocoat composition comprising: a) a biosticker composition of the present disclosure; and b) at least one microbial culture, wherein the microbial culture has microbial load in the range of 10⁷-10¹⁰ in 100 ml of the biocoat composition. The microbial culture had microbial load in the range of 10⁷-10¹⁰ in 100 ml of the biocoat composition.

The present example exemplifies the process for preparing the biocoat composition comprising biosticker composition C7. The said biocoat composition was prepared by the following steps: the biosticker composition C7 was mixed with at least one microbial culture at a ratio of 1:1 (50 ml Biosticker+50 ml Microbes), 1:2 (33.33 ml Biosticker+66.67 ml culture), 1:3 (25 ml Biosticker+75 ml culture), 1:5 (16.67 ml biosticker+83.3 ml culture), 1:6 (14.3 ml Biosticker+85.7 ml Culture), 2:1 (66.67 ml Biosticker+33.33 ml culture), 2:3 (40 ml Biosticker+60 ml culture), 2:5 (28.5 ml Biosticker+71.5 ml culture), 3:1 (75 ml Biosticker+25 ml culture), 3:2 (60 ml Biosticker+40 ml culture), 3:5 (37.5 ml Biosticker+62.5 ml culture) under sterile conditions. The microbial culture had microbial load in the range of 10⁷-10¹⁰ in 100 ml of the biocoat composition.

Similarly, all the biosticker composition C1-C6 as per the Table 1 of Example 1 can be used for preparing the biocoat composition by following the process as described above. Nevertheless, it can be contemplated that the person skilled in the art can use different biosticker compositions with the constituents in the disclosed weight percentages, to arrive at the biocoat composition of the present disclosure.

Process for preparing the biocoat composition comprising the biosticker composition and microbial culture: The present disclosure further relates to another biocoat composition comprising: a) a biosticker composition; b) at least one microbial culture; and c) at least one pesticide, wherein the microbial culture had microbial load in the range of 10⁷-10¹⁰ in 100 ml of the biocoat composition.

The present example exemplifies the process for preparing the biocoat composition comprising biosticker composition C7. The said biocoat composition was prepared by the following steps: the biosticker composition C7 was mixed with at least one microbial culture (Table 3) in a ratio in the range of 1:1-1:6 (Biosticker:Microbial culture) under sterile conditions to obtain a solution. The said solution was then further mixed with the chemical pesticide to prepare the biocoat composition for seed coating.

Similarly, all the biosticker composition C1-C6 as per the Table 1 of Example 1 can be used for preparing the biocoat composition by following the process as described above. Nevertheless, it can be contemplated that the person skilled in the art can use different biosticker compositions with the constituents in the disclosed weight percentages, to arrive at the biocoat composition of the present disclosure.

Table 3 of the present disclosure depicts the list of microbial cultures used in the process for obtaining biocoat composition as disclosed herein.

TABLE 3

| S. No. | Name of the microbes | Accession Number |
|---|---|---|
| I | Rhizobium spp | MTCC 25075 |
| II | Azospirillum spp | MTCC 25052 |
| III | Azotobacter spp | MTCC 25051 |
| IV | Bacillus subtilis | MTCC 5728 |
| V | Phosphate solubilizing bacteria | MTCC 25049 |
| VI | Trichoderma viride | MTCC 25012 |
| VII | Paecilomyces spp | ITCC 6887 |
| VIII | Pochonia spp | ITCC 6988 |

Table 4 of the present disclosure depicts the list of pesticide which were used to arrive at the biocoat composition as disclosed herein.

TABLE 4

| Pesticide | Examples |
|---|---|
| Insecticides | Chlorpyriphos, Diazinon, Imidacloprid, Lindane, Permethrin, |
| | Tefluthrin, Thiamethoxam, and Cyantranilliprole |
| Fungicides | Captan (N-trichloromethylthio-4-cyclohexene-1,2- dicarboximide), |
| | Carboxin (5,6-dihydro-2-methyl-N-phenyl-1,4-oxathiin-3- |
| | carboxamide), Difenoconazole (cis,trans-3-chloro-4-[4-methyl-2-(1H- |
| | 1,2,4- triazol-1-ulmethyl)-1,3-dioxolan-2-yl]phenyl 4- chlorophenyl |
| | ether), Imazalil (+)-allyl 1-(2,4-dichlorophenyl)-2-imidazol-1- ylethyl |
| | ether), Mancozeb (Zinc Manganese ethylenebisdithiocarbamate), |
| | Maneb (Manganese ethylenebisdithiocarbamate), Metalaxyl (methyl N- |
| | (2-methoxyacetyl)-N-(2,6-xylyl)-Dlalaninate), PCNB |
| | (Pentachloronitrobenzene), Tebuconazole (RS)-1-(4-chlorophenyl)4,4- |
| | dimethyl-3-(1H1,2,4-triazol-1-ulmethyl) pentan-3-ol), Thiabendazole |
| | (2-(4-Thiazolyl)-benzimidazole), Thiram |
| | (Tetramethylthiuramdisulfide), Triadimenol (1RS, 2RS; 1RS, 2SR)-1- |
| | (r-chlorophenoxy)-3,3- dimethyl-1-(1H-1,2,4-triazol-1-yl) butan-2-ol), |
| | Triticonazole (+)-(E)-5-(4-chlorobenzylidene)-2-dimethyl-1- (1H- |
| | 1,2,4-triazol-1-ylmethyl) cyclopentanol), Fludioxonil, Mefenoxam, and |
| | Metalaxyl |
| Nematicide | Telone II, Chlor-O-Pic, Telone C-17, Telone C-35, Telone EC, InLine, |
| | PicClor-60, Vydate (L, C, LV), Nimitz, Velum Prime, Mocap 15 G, |
| | Mocap EC, Movento, Counter 20 G, Salibro**, Paecilomyces lilacinus, |
| | and Bacillus spp. |
| Herbicides | 2,4-Dichlorophenoxy Acetic Acid, Alachlor, Anilophos, Atrazine, |
| | Azimsulfuron, Bensulfuron Methyl, Bispyribac Sodium, Butachlor, |
| | Carfentazone Ethyl, Chlorimuron ethyl, Chlormequat Chloride (CCC), |
| | Chlorpropham, Clodinafop-propargyl, Clomazone, Cyhalofop-butyl, |
| | Dazomet, Diclofop-Methyl, Diuron, Ethoxysulfuron, Fenoxaprop-p- |
| | ethyl, Fluazifop-p-butyl, Fluchloralin, Flufenacet, Glufosinate |
| | Ammonium, Glyphosate, Hexazinone, Imazamox, Imazethapyr, |
| | Isoproturon, Linuron, Mepiquate Chloride, Mesosulfuron Methyl + |
| | Iodosulfuron Methyl Sodium, Metaflumizone, Methabenzthiazuron, |
| | Methyl Chlorophenoxy Acetic Acid (MCPA), Metolachlor, Metribuzin, |
| | Metsulfuron Methyl, Orthosulfamuron, Oxadiargyl, Oxadiazon, |
| | Oxyfluorfen, Paraquat dichloride, Pendimethalin, Pinoxaden, |
| | Pretilachlor, Propanil, Propaquizafop, Pyrazosulfuron ethyl, Pyrithiobac |
| | sodium, Quizalofop ethyl, Quizalofop-P-tefuryl, Sirmate, |
| | Sulfosulfuron, and Trifluralin |

It can be contemplated that a person skilled in the art can arrive at different biocoat combinations comprising: biosticker composition, microbial culture or combination of microbial cultures as depicted in Table 3, and/or pesticide or combination of pesticide as depicted in Table 4, by the process as disclosed above.

### Example 4

### Biocoat Compositions for Coating the Seeds

The present disclosure provides different biocoat compositions comprising: (a) combinations of biosticker composition and microbial culture; (b) combinations of biosticker composition, microbial culture, and chemical pesticides. The criticality of the present disclosure lies in the ratio of biosticker composition to the microbical culture, wherein the biosticker composition to the the microbial culture was present at a ratio of 1:1 (50 ml Biosticker+50 ml Microbes), 1:2 (33.33 ml Biosticker+66.67 ml culture), 1:3 (25 ml Biosticker+75 ml culture), 1:5 (16.67 ml biosticker+83.3 ml culture), 1:6 (14.3 ml Biosticker+85.7 ml Culture), 2:1 (66.67 ml Biosticker+33.33 ml culture), 2:3 (40 ml Biosticker+60 ml culture), 2:5 (28.5 ml Biosticker+71.5 ml culture), 3:1 (75 ml Biosticker+25 ml culture), 3:2 (60 ml Biosticker+40 ml culture), 3:5 (37.5 ml Biosticker+62.5 ml culture). Therefore, it can be contemplated that a person skilled in the art can combine prepare the biocoat composition by combining the biosticker composition in the range of 14.3 ml-75 ml, and the microbial culture was present in the range of 25 ml-85.7 ml.

Table 5 provides different biocoat compositions having a combination of different biosticker compositions and microbial culture.

Constituents of the biocoat
formulation along with the
biosticker compositions

| | Name of the | | |
|---|---|---|---|
| | Microbial | | |
| Biocoat | culture(s) | | |
| formulation | (Microbial | | Seed |
| (100 ml) | load 10⁷-10¹⁰) | Pesticide | Material |
| F1 | Bacillus | | Wheat |
| | subtilus | | |
| F2 | Trichoderma | | Paddy |
| | viride + | | |
| | Azospirillum | | |
| | spp + | | |
| | Burkholderia | | |
| | vietnamese | | |
| F3 | Trichoderma | | Chickpea |
| | viride + | | |
| | Bacillus | | |
| | aerius | | |
| F4 | Trichoderma | | Corn |
| | viride + | | |
| | Bacillus | | |
| | pumilus + | | |
| | Burkholderia | | |
| | vietnamese | | |
| F5 | Trichoderma | | Corn |
| | viride + | | |
| | Azospirillum | | |
| | spp + | | |
| | Burkholderia | | |
| | vietnamese | | |
| F6 | Vesicular- | | Okra |
| | arbuscular | | |
| | mycorrhiza + | | |
| | Bacillus | | |
| | subtilus + | | |
| | Bacillus | | |
| | aerius + | | |
| | Bacillus | | |
| | paramycoides | | |
| F7 | Bacillus | Metalaxyl | Tomato |
| | pumilus + | | |
| | Bacillus | | |
| | aerius | | |
| F8 | Bacillus | Cyantraniliprole | Corn |
| | pumilus + | | |
| | Bacillus | | |
| | subtilus | | |
| F9 | Bacillus | Thiamethoxam | Corn |
| | subtilus + | | |
| | bacillus | | |
| | pumilus + | | |
| | Bacillus | | |
| | aerius | | |
| F10 | Bacillus | Thiamethoxam + | Corn |
| | pumilus + | Metalaxyl + | |
| | Bacillus | | |
| | aerius + | | |
| | Azotobacter | | |
| | chroococcum | | |

Table 5 shows different biocoat compositions F1-F10 that can be used for coating seeds. The bocoat compositions F1-F10 comprised the biosticker compositions as disclosed in the present disclosure. For instance, the biocoat composition F1 comprised biocoating composition C7 and Bacillus subtilus, that was used for coating wheat seeds. The weight ratio of the biosticker composition C7 to Bacillus subtilus ranged between 1:1 to 1:6. However, it can be contemplated that the other biosticker compositions as described above in Table 1 can also be used for arriving at the biocoat composition F1. Further the biocoat composition can be used for coating other seed materials such as corn, paddy, soyabean, chickpea, okra, tomato, sugarbeet. Similarly, the other biocoat compositions F2-F10 comprised any one of the biosticker compositions as disclosed in Table 4. Apart from the mentioned seed material, the biocoat compositions F1-F10 can be used for coating seed materials selected from the group consisting of rice (Oryza sativa), wheat, bread wheat (Triticum aestivum), corn (Zea mays), maize (Zea mays), barley (Hordeum vulgare), Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor), red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), gingelly (Sesamum indicum), sunflower (Helianthus spp.), groundnut (Apios americana), mustard (Brassica juncea), safflower (Carthamus tinctorius), and castor (Ricinus communis), fibre crops are selected form the group consisting of cotton (Gossypium spp.), and jute (Corchorus olitorius), tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), sweet sorghum (Sorghum spp.), and sugar beet (Beta vulgaris), fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala).

In the biocoat composition F1-F10, the biosticker composition was used in a range of 14.3-75 wt %, and the microbial culture was used in a range of 25-85.7 wt %. It is noteworthy to mention here that the the weight ratio of biosticker composition to the microbial culture is critical for arriving at the biocoat composition, and the weight ratio ranged between 1:1 to 1:6. The microbial culture had microbial load in the range of 10⁷-10¹⁰ in 100 ml of the biocoat composition. The biocoat compositions F1-F6 had a shelf life of 24 months, 12 months, 20 months, 19 months, 19 months, and 8 months, respectively.

In some of the biocoat compositions such as F7, F8, F9, and F10, a combination of biosticker composition, microbial culture and pesticides were used. In the biocoat compositions F7-F10, the chemical pesticides were used in the range of 0.5-10%, which was added in grams or volumes depending on the form of the pesticide used. Apart from the pesticides mentioned in Table 5, a person skilled in the art can use any of the chemical pesticides disclosed in the present disclosure.

The role of the presence of chemical pesticides in the biocoat composition is to provide the protection to seeds from the diseases, whereas, the microbes is used for providing nutritional benefits and growth of the seeds. Therefore, it can be inferred that the presence of the biosticker composition, microbial culture in the disclosed weight ratio range of 1:1 to 1:6 is crucial for arriving at the biocoat composition that is used for coating seeds. Further, the presence of the chemical pesticides in the disclosed weight percentage range (0.5-10%) along with the biosticker composition and microbial culture is important for arriving at the biocoat composition of the present disclosure.

### Example 5

### Process for Coating Seed Material

The present example relates to the process followed for the coating of seed material with any of the biocoat compositions as obtained in the Example 3 of the present disclosure. The said biocoat composition was added with the seed material to form a thin film over the seed material as coating. The coated seeds were dried under sterile condition and stored under airtight sterile containers. The coated seeds were evaluated on the basis of colony forming units (CFU). The parameters that were studied included pH of biosticker composition, appearance of coated seeds and non-coated seeds, weight of 10 coated seeds, seed germination of the coated seeds, effect of coating on plant morphological and yield attribute and shelf-life for determination of survivability duration.

Depending upon different aspects including, the microbial load on the seed surface, surface covering percentage, drying time, seed germination and plant growth, varying coating volumes (ranging between 5 ml/1000 g to 100 ml/1000 g) of the biocoat solution were used to coat the “Super-30” corn seeds (commercial variety procured from the local market) in the present example. The microbial culture used in the biocoat solution for coating corn seeds was Bacillus subtilis (MTCC 5728). Table 6 shows the assessment of the microbial load over the coated seeds, with varying coating volumes used for corn seeds. The coating process was also applied to the following seed materials, namely, wheat (Raj 3077), soybean (Durga—JS 72-280), chickpea (Karnal Chana—Local variety in Haryana), paddy (Naina—CSR-36), Okra (akra anamika—Sweta Seeds, Delhi), sugar beet/fodder beet (Samridhan Poshan), for the assessment of microbial load over the coated seeds.

TABLE 6

| Coating volume of biocoat | | Percentage of seed | |
|---|---|---|---|
| solution used for coating | | surface covered | |
| “Super-30” corn | Microbial | by biocoat | Seed drying time |
| seeds (ml/1000 g) | load | solution (%) | after coating |
| 5 | 10⁶ | 65-70 | Instant dry |
| 10 | 10⁶ | 80-85 | Instant dry |
| 20 | 10⁷ | 90-95 | 30 minutes |
| 100 | 10⁸ | 100 | 1.5-2 hours |

As per the Table 6, four variants of biocoat coating volumes were used for coating the corn seeds, wherein 10⁸ of microbial load, 100% surface coverage was observed with the drying time of 1.5-2 hours in case of seeds coated with the coating volume of 100 ml/1000 g of corn seeds. However, a considerably less drying time of 30 minutes with 90-95% surface coverage and 10⁷ microbial load was observed in case of corn seeds coated with the coating volume of 20 ml/1000 g. The biocoat coating volume of 5 ml/1000 g and 10 ml/1000 g showed 65-70% and 80-85% seed surface coverage respectively, with same microbial load of 10⁶ and instant drying of coated corn seeds. Therefore, it could be inferred that, the coating volumes (ranging between 5 ml/1000 g to 100 ml/1000 g) can be used for the purpose of coating of seeds as per the present disclosure.

Corn seeds as coated in the present example described herein were found to have a shelf-life of more than 2 years, i.e. the microbe (Bacillus subtilis-MTCC 5728) were found to be viable for approximately more than 2 years after the coating process. The extended viability of the microbe would thus play a pivotal role upon sowing of the seeds. However, in case of coating the seed material with biocoat composition comprising non-sporulating bacteria, shelf-life of only 6 months was observed. Further, in case of soybean seeds, poor germination was observed in 15 coated seeds which were coated using non-sporulating bacteria. Therefore, it can be inferred that, the microbial load in the range of 10⁷-10¹⁰ in 100 ml of the biocoat composition, is crucial to arrive at the desired biocoat composition for the coating of seeds as per the present disclosure.

### Example 6

### Comparative Example

The present example demonstrates the criticality of coating the seeds with the biocoat compositions of the present disclosure. For this purpose, the pot culture study of seeds coated with the biocoat compositions of the present disclosure was done. The biocoat seeds were compared with the non-coated seeds, based on the following characteristic features, namely, germination percentage, pre-emergence percentage, post-emergence damping off, total damping off percent observed in 20 days after sowing (DAS), mean shoot length observed in 20 DAS, mean root length observed in 20 DAS, and seedling vigour observed in 20 DAS. The results are shown in Table 7.

TABLE 7

| | | | Pre- | Post- | Total | Mean Shoot | Mean Root | |
|---|---|---|---|---|---|---|---|---|
| Microbial | | | emergence | emergence | damping | length | length | Seedling |
| Seed coating | | Germination % | damping | damping | off (%) | (cm) | (cm) | vigour |
| Details | Treatments | (10 DAS) | off % | off % | (20 DAS) | (20 DAS) | (20 DAS) | (20 DAS) |
| Seeds coated | Sterile Soil + | 86.67 | | | | 33.50 | 2.38 | 3229.20 |
| with F1 | Untreated seed | | | | | | | |
| composition | Sterile Soil + | 98.33 | | | | 31.90 | 6.00 | 3726.83 |
| | Coated seed | | | | | | | |
| | Sick soil + | 78.33 | 21.67 | 69.15 | 90.82 | 34.60 | 3.64 | 2995.47 |
| | Untreated Seed | | | | | | | |
| | Sick soil + | 86.67 | 13.33 | 59.19 | 72.52 | 29.60 | 4.46 | 3008.63 |
| | Coated seed | | | | | | | |
| Seeds coated | Sterile Soil + | 80.00 | | | | 48.80 | 5.20 | 4500.00 |
| with F1 | Untreated seed | | | | | | | |
| composition F2 | Sterile Soil + | 100.00 | | | | 47.20 | 10.40 | 5760.00 |
| | Coated seed | | | | | | | |
| | Sick soil + | 81.67 | 13.33 | 55.29 | 68.62 | 22.50 | 7.20 | 2376.00 |
| | Untreated Seed | | | | | | | |

TABLE 8

| | | | | | Soil | Nitrogen | Phosphorus | Phosphorus |
|---|---|---|---|---|---|---|---|---|
| | | | Mean Shoot | Mean Root | available | uptake | available | uptake by |
| Microbial | | Germination | length at | length at | nitrogen | by plant | in soil | plant |
| Seed coating | | (10 DAS) | (35 DAS) | (35 DAS) | (12 DAS) | (35 DAS) | (12 DAS) | (35 DAS) |
| Details | Treatments | Percentage | (cm) | (cm) | % | % | Kg/ha | Mg/kg |
| Seeds coated | Sick soil + | 76.6 | 30 | 5.7 | 0.19 | 4.03 | 1556 | 7.3 |
| with F10 | Untreated Seed | | | | | | | |
| composition | Sick soil + | 100 | 33 | 9.7 | 0.247 | 4.47 | 4587.3 | 9.6 |
| | Coated seed | | | | | | | |
| Seeds coated | Sick soil + | 76.6 | 30 | 5.7 | 0.19 | 4.03 | 1556 | 7.3 |
| with chemical | Untreated Seed | | | | | | | |
| pesticide only | Sick soil + | 90.0 | 31 | 6.3 | 0.219 | 4.12 | 3675.2 | 9.6 |
| (Nonn-working | Coated seed | | | | | | | |
| Composition) | | | | | | | | |

Referring to Table 7 it can be observed that the germination percentage was higher in the seeds coated with the biocoat composition of the present disclosure as compared to non-coated seeds in both sterile and infected soil. Further, low damping off was observed in coated seeds with biocoat compositions F1 and F2 as compared to non-coated seed. The shoot length was observed higher in seeds coated with the biocoat compositions F1 and F2, as compared to non-coated seeds. The root length was observed better in all type of seeds coated with biocoat composition F1 and F2. Although only results of the biocoat compositions F1 and F2 are compared with the non-coated seeds, however, the results extend to all the biocoat compositions F1-F6.

Further, the germination percentage was reported higher in seeds coated with the biocoat composition comprising biosticker composition, microbial culture and chemical pesticides (for example F7-F10), as compared to the non-coated seeds, or seeds only coated with the chemical pesticides. Parameters like shoot length, root length, nitrogen uptake by plant, phosphorus uptake by plant were observed higher in seeds coated with the biocoat compositions F7-F10 in comparison to non-coated seeds. Moreover, damping off was also low in seeds coated with the biocoat compositions F7-F10, as compared to non-coated seeds.

Referring to Table 8, it can be inferred that all the parameters like seed germination shoot length, root length, soil available nitrogen, soil available phosphorus, nitrogen uptake by plant, phosphorus uptake by plants were higher in the seeds coated with the biocoat composition F10. In contrast, the nitrogen uptake by plant was lower in the seeds coated with the chemical pesticide only. Hence, this is considered as a non-working example.

Therefore, it can be inferred that the coating seeds with the biocoat composition (biosticker compositin+microbial culture), or biocoat composition (biosticker compositin+microbial culture+chemical pesticide) of the present disclosure exhibits better germination shoot length, root length, soil available nitrogen, soil available phosphorus, nitrogen uptake by plant, phosphorus uptake by plants as compared to the non-coated seeds or seeds coated only with chemical pesticides. Thus, from the above it can be inferred that the components of the biocoat composition of the present disclosure works synergistically resulting in higher germination percentage and better growth as compared to the non-coated seeds or seeds only coated with the chemical pesticides.

### ADVANTAGES OF THE PRESENT DISCLOSURE

The present disclosure discloses a biosticker and biocoat composition for the use in coating of seed material. The process of coating a seed material using the present seed coating composition (biocoat) is also disclosed herein. Unlike the conventional compositions employed for coating of seeds, the disclosed composition is biodegradable and thereby supports sustainable agricultural practices. Further, the biocoat composition as disclosed herein displays a longer shelf with efficient binding ability to the seed material. It is also observed to possess non-inhibitory nature to the microbes which may support the growth and nutritional uptake of the coated seeds. Also, the present biocoat composition additionally provides enhanced germination percentage, nutrient uptake, seedling vigour, growth and lowered damping off in coated seeds on comparison to non-coated seeds. The biocoat composition as described herein performs specific functions such as nitrogen fixation, phosphate solubilization, plant-growth promotion, biological control of plant pathogens, and biological control of pests. Overall, the disclosed biocoat composition could be considered as an efficient, safe and effective seed coating material

1. A composition comprising:
a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof;
b) at least one starch having a weight percentage in a range of 2-15% with respect to the composition;
c) organic acid having a weight percentage in a range of 0.01-1% with respect to the composition, wherein the organic acid is selected from the group consisting of citric acid, tartaric acid, ascorbic acid and combinations thereof; and
d) at least one solvent having a weight percentage in a range of 1-10% with respect to the composition, wherein the solvent is selected from the group consisting of glycerol, propylene glycol, and combinations thereof.

2. The composition as claimed in claim 1, wherein the polymer is selected from the group consisting of polyvinyl alcohol, polyvinyl acetate, and polyvinyl pyrrolidones.

3. The composition as claimed in claim 1, wherein the starch is selected from the group consisting of corn starch, wheat starch, and rice starch.

4. The composition as claimed in claim 1, wherein the organic acid is citric acid.

5. The composition as claimed in claim 1, wherein the solvent is glycerol.

6. The composition as claimed in claim 1, wherein the composition further comprises at least one excipient.

7. The composition as claimed in claim 6, wherein the excipient is selected from the group consisting of gum acacia having weight percentage in the range of 0.5-10% with respect to the composition, xanthan gum having weight percentage in the range of 0.5-10% with respect to the composition, polyethylene glycol having weight percentage in the range of 0.5-10% with respect to the composition, natural or synthetic colour having weight percentage in the range of 0.05-0.1% with respect to the composition, and combinations thereof.

8. A composition comprising:
a) polyvinyl alcohol having a weight percentage in the range of 2-10% with respect to the composition;
b) corn starch having a weight percentage in the range of 2-10% with respect to the composition;
c) citric acid having a weight percentage in the range of 0.1-0.91% with respect to the composition;
d) glycerol having a weight percentage in the range of 2-10% with respect to the composition;
e) gum acacia having a weight percentage in the range of 2-10% with respect to the composition;
f) xanthan gum having a weight percentage in the range of 2-10% with respect to the composition; and
g) polyethylene glycol having a weight percentage in the range of 2-10% with respect to the composition.

9. A process for preparing the composition as claimed in claim 1, said process comprising:
a) contacting at least one polymer with water to obtain a first mixture, wherein the polymer having a weight percentage in a range of 1-10% with respect to the composition is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof;
b) contacting the first mixture with at least one starch to obtain a second mixture, wherein the starch having a weight percentage in a range of 2-15% with respect to the composition is selected from the group consisting of corn starch, wheat starch, rice starch, and combinations thereof;
c) mixing the second mixture with organic acid to obtain a third mixture, wherein the organic acid having a weight percentage in a range of 0.01-1% with respect to the composition is selected from the group consisting of citric acid, tartaric acid, ascorbic acid and combinations thereof; and
d) contacting the third mixture with at least one solvent to obtain the composition, wherein the solvent having a weight percentage in a range of 1-10% with respect to the composition is selected from the group consisting of glycerol, propylene glycol, and combinations thereof, and wherein the composition has a pH in a range of 2-5.

10. The process as claimed in claim 9, wherein contacting the at least one polymer with water is carried out at a temperature in the range of 80-95° C. to obtain the first mixture, and wherein contacting the first mixture with the at least one starch is carried out at a temperature in the range of 80-85° C. to obtain the second mixture, and wherein mixing the second mixture with the at least one citric acid is carried out at a temperature in the range of 60-70° C. to obtain the third mixture, and wherein contacting the third mixture with the at least one solvent is carried out at a temperature in the range of 50-60° C. to obtain the composition.

11. The process as claimed in claim 9, wherein the process further comprises contacting the third mixture with the excipient selected from the group consisting of gum acacia, xanthan gum, polyethylene glycol, natural or synthetic colour, and combinations thereof, to obtain the composition.

12. A biocoat composition comprising:
(i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof;
ii) at least one microbial culture; and
iii) at least one pesticide.

13. A biocoat composition comprising:
(i) a composition comprising: a) at least one polymer having a weight percentage in a range of 1-10% with respect to the composition, wherein the polymer is selected from the group consisting of polyvinyl alcohol, polyvinyl alcohol copolymers, polyvinyl acetate, polyvinyl acetate copolymers, polyvinyl pyrrolidones, vinylidene chloride, vinylidene chloride copolymers, and combinations thereof; b) at least one starch having a weight percentage in a range of 2-15% with respect to the composition; c) at least one organic acid having a weight percentage in a range of 0.01-1% with respect to the composition, wherein the organic acid is selected from the group consisting of citric acid, tartaric acid, ascorbic acid and combinations thereof; d) at least one solvent having a weight percentage in a range of 1-10% with respect to the composition, wherein the solvent is selected from the group consisting of glycerol, propylene glycol, and combinations thereof; e) gum acacia having a weight percentage in the range of 0.5-10% with respect to the composition; f) xanthan gum having a weight percentage in the range of 0.5-10% with respect to the composition; g) polyethylene glycol having a weight percentage in the range of 0.5-10% with respect to the composition;
ii) at least one microbial culture; and
iii) at least one pesticide.

14. A biocoat composition comprising:
a) the composition as claimed in claim 1;
b) at least one microbial culture.

15. The biocoat composition as claimed in claim 12, wherein the microbial culture is selected from the group consisting of Rhizobium spp. (Rhizobium japonicum), Azospirillum spp., Azotobacter spp. (Azotobacter chroococcum), Bacillus spp. (Bacillus aerius, Bacillus paramycoides, Bacillus subtilus, Bacillus thuringiensis var. kurtaski, Bacillus polymyxa), Trichoderma spp. (Trichoderma viride, Trichoderma harzianum, Trichoderma asperellum), Vesicular-Arbuscular Mycorrhiza (VAM), Paecilomyces spp. (Paecilomyces lilacinus), Pochonia spp., Acetobacter diazotrophicus, celluloso microbium funksi, Glomus intraradices, Pseudomonas fluorescens, Ampelomyces quisqualis, Verticillium lecanii, Hirsutella thompsonii, Beauveria bassiana, Metarhizium anisopliae, Verticillium chlamydosporia, plant growth-promoting rhizobacteria (PGPR), phosphate-solubilizing bacteria, potash mobilising bacteria, zinc solubilizing bacteria, biofungicide, and combinations thereof.

16. The biocoat composition as claimed in claim 12, wherein the microbial culture has microbial load in the range of 10⁷-10¹⁰ in 100 ml of the biocoat composition.

17. The biocoat composition as claimed in claim 12, wherein the pesticide is selected from the group consisting of insecticides, fungicides, nematicide, herbicides, and combinations thereof.

18. The biocoat composition as claimed in claim 17, wherein the insecticides are selected from the group consisting of Chlorpyriphos, Diazinon, Imidacloprid, Lindane, Permethrin, Tefluthrin, Thiamethoxam, and Cyantranilliprole; the fungicides are selected from the group consisting of Captan (N-trichloromethylthio-4-cyclohexene-1,2-dicarboximide), Carboxin (5,6-dihydro-2-methyl-N-phenyl-1,4-oxathiin-3-carboxamide), Difenoconazole (cis,trans-3-chloro-4-[4-methyl-2-(1H-1,2,4-triazol-1-ulmethyl)-1,3-dioxolan-2-yl]phenyl 4-chlorophenyl ether), Imazalil (+)-allyl 1-(2,4-dichlorophenyl)-2-imidazol-1-ylethyl ether), Mancozeb (Zinc Manganese ethylenebisdithiocarbamate), Maneb (Manganese ethylenebisdithiocarbamate), Metalaxyl (methyl N-(2-methoxyacetyl)-N-(2,6-xylyl)-Dlalaninate), PCNB (Pentachloronitrobenzene), Tebuconazole (RS)-1-(4-chlorophenyl)4,4-dimethyl-3-(1H1,2,4-triazol-1-ulmethyl) pentan-3-ol), Thiabendazole (2-(4-Thiazolyl)-benzimidazole), Thiram (Tetramethylthiuramdisulfide), Triadimenol (1RS,2RS;1RS,2SR)-1-(r-chlorophenoxy)-3,3-dimethyl-1-(1H-1,2,4-triazol-1-yl) butan-2-ol), Triticonazole (+)-(E)-5-(4-chlorobenzylidene)-2-dimethyl-1-(1H-1,2,4-triazol-1-ylmethyl) cyclopentanol), Fludioxonil, Mefenoxam, and Metalaxyl; the nematicides are selected from the group consisting of Telone II, Chlor-O-Pic, Telone C-17, Telone C-35, Telone EC, InLine, PicClor-60, Vydate (L, C, LV), Nimitz, Velum Prime, Mocap 15G, Mocap EC, Movento, Counter 20G, Salibro**, Paecilomyces lilacinus, and Bacillus spp.; the herbicides are selected from the group consisting of 2,4-Dichlorophenoxy Acetic Acid, Alachlor, Anilophos, Atrazine, Azimsulfuron, Bensulfuron Methyl, Bispyribac Sodium, Butachlor, Carfentazone Ethyl, Chlorimuron ethyl, Chlormequat Chloride (CCC), Chlorpropham, Clodinafop-propargyl, Clomazone, Cyhalofop-butyl, Dazomet, Diclofop-Methyl, Diuron, Ethoxysulfuron, Fenoxaprop-p-ethyl, Fluazifop-p-butyl, Fluchloralin, Flufenacet, Glufosinate Ammonium, Glyphosate, Hexazinone, Imazamox, Imazethapyr, Isoproturon, Linuron, Mepiquate Chloride, Mesosulfuron Methyl+Iodosulfuron Methyl Sodium, Metaflumizone, Methabenzthiazuron, Methyl Chlorophenoxy Acetic Acid (MCPA), Metolachlor, Metribuzin, Metsulfuron Methyl, Orthosulfamuron, Oxadiargyl, Oxadiazon, Oxyfluorfen, Paraquat dichloride, Pendimethalin, Pinoxaden, Pretilachlor, Propanil, Propaquizafop, Pyrazosulfuron ethyl, Pyrithiobac sodium, Quizalofop ethyl, Quizalofop-P-tefuryl, Sirmate, Sulfosulfuron, and Trifluralin.

19. The biocoat composition as claimed claim 12, wherein the composition further comprises natural or synthetic colour having a weight percentage in the range of 0.05-0.1% with respect to the composition.

20. The biocoat composition as claimed claim 12, wherein the pesticide has a weight percentage in the range of 0.5-15% with respect to the composition.

21. A process for coating a seed material, said process comprising:
a) mixing the composition as claimed in claim 1 with the microbial culture and the pesticide to obtain the biocoat composition; and
b) adding the biocoat composition to a seed material followed by drying for coating the seed material, wherein adding the biocoat composition to the seed material in a volume in the range of 5 ml/1000 g-100 ml/1000 g seed.

22. The process as claimed in claim 21, wherein adding the biocoat composition to a seed material followed by drying for coating the seed material is done under sterile condition.

23. The process as claimed in claim 21, wherein the seed material is selected from the group consisting of cereals, millets, pulses, oil seeds, fibre crops, vegetable crops, sugar crops, and forage crops.

24. The process as claimed in claim 23, wherein the cereals are selected form the group consisting of rice (Oryza sativa), wheat, bread wheat (Triticum aestivum), corn (Zea mays), maize (Zea mays), barley (Hordeum vulgare); and wherein the millets are selected form the group consisting of Panicum miliaceum, Eleusine coracana, Setaria italica, Pennisetum glaucum, Panicum sumatrense, Panicum italicum, Paspalum scrobiculatum, Echinochloa frumentacea, and sorghum (Sorghum bicolor); and wherein the pulses are selected form the group consisting of red gram (Cajanus cajan), black gram (Vigna mungo), green gram (Vigna radiata), bengal gram (Cicer arietinum), lab lab (Lablab purpureus), soybean (Glycine max), and cow pea (Vigna unguiculata), and wherein the oil seeds are selected form the group consisting of gingelly (Sesamum indicum), sunflower (Helianthus spp.), groundnut (Apios americana), mustard (Brassica juncea), safflower (Carthamus tinctorius), and castor (Ricinus communis), and wherein the fibre crops are selected form the group consisting of cotton (Gossypium spp.), and jute (Corchorus olitorius), and wherein the vegetables are selected form the group consisting of tomatoes (Solanum lycopersicum), brinjal (Solanum melongena), okra (Abelmoschus esculentus), chilly (Capsicum spp.), ash gourd (Benincasa hispida), ribbed gourd (Luffa acutangula), bitter gourd (Momordica charantia), pumpkin (Cucurbita maxima), and onion (Allium cepa), and wherein the sugar crops are selected from the group consisting of sweet sorghum (Sorghum spp.), and sugar beet (Beta vulgaris), and wherein the forage crops are selected form the group consisting of fodder cholam (Sorghum bicolor (L.) Moench), fodder cumbu (Pennisetum glaucum), fodder maize (Zea mays L.), cumbu napier hybrid (Cenchrus purpureus), guinea grass (Megathyrsus maximus), kolukattai pullu (Cenchrus Ciliaris), kudirai masal (Medicago sativa), velimasal (Desmanthus virgatus), fodder cowpea (Vigna unguiculata), muyal masal (Stylosanthes scabra), and soundal (Leucaena leucocephala).

25. The process as claimed in claim 21, wherein the microbial culture has microbial load in the range of 10⁷-10¹⁰ in 100 ml of the biocoat composition.
